(19) United States
(12) Patent Application Publication
(10) Pub. No.: US 2006/0135449 A1
(43) Pub. Date: Jun. 22, 2006

(54) Decoy compositions for treating and preventing brain diseases and disorders

(76) Inventors: Yoshiki Sawa, Osaka (JP); Ryuichi Morishita, Osaka (JP); Yasufumi Kaneda, Osaka (JP); Hikaru Matsuda, Osaka (JP); Toshiki Yoshimine, Osaka (JP)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 10/509,799

(22) PCT Filed: Mar. 29, 2002
(86) PCT No.: PCT/JP02/03239

§ 371 Date: Jul. 15, 2005

(51) Int. Cl. A61K 48/00; A61K 9/127

(52) U.S. Cl. 514/44; 424/450

(57) ABSTRACT

The present invention provides introduction of NF-κB decoy oligodeoxynucleotide into rat cranial nerve through a carotid artery during global brain ischemia. Polymerase chain reaction demonstrated that one hour after global brain ischemia, transfected NF-κB decoy oligodeoxynucleotide effectively suppressed expression of tumor necrosis factor α, interleukin 1β and intracellular adhesion molecule 1 messenger RNAs. Terminal deoxynucleotidyl transferase-mediated deoxyuridine nick-end labeling staining and immunohistochemistry using microtubule-associated protein 2 demonstrated that transfected NF-κB decoy oligodeoxynucleotide significantly attenuated neuronal damage seven days after global brain ischemia. Therapeutic transfection of NF-κB decoy oligodeoxynucleotide during brain ischemia may be effective for attenuation of neuronal damage, suggesting a strategy for protecting the cerebrum from global ischemia.

### TECHNICAL FIELD

The present invention relates to a composition comprising a compound (e.g., a nucleic acid and a homolog thereof) which specifically binds to a site to which a transcriptional regulatory factor binds, and a method of using the same. More particularly, the present invention relates to a composition for treating a cerebral ischemic disorder, comprising a decoy compound (e.g., a nuclear factor κB (NF-κB) decoy), and a method of using the same. The present invention also provides a method for carrying out gene transfection into the brain by administration via a route other than the brain, and a composition for the same.

### BACKGROUND ART

A variety of diseases including asthma, cancers, heart diseases, aneurysms, autoimmune diseases, and viral infections manifest varying symptoms and signs and yet it has been suggested that an abnormal expression (an overexpression or underexpression) of one or a few proteins is a major etiologic factor in many cases. In general, the expression of those proteins is controlled by a variety of transcriptional regulatory factors such as transcription activating factors and transcription suppressing genes.

NF-κB is one of such transcriptional regulatory factors for genes encoding gene products important for inflammation and immune (Baeuerle P A. et al., Annu Rev Immunol. 1994; 12:141-79). NF-κB responds to various extracellular signals and migrates from the cytoplasm to the nucleus, and plays a pivotal role in the coordinated transactivation of several cytokines and adhesion molecule genes. Cooper et al. demonstrated a time-dependent increase in the DNA binding activity of NF-κB, which had a peak three days before rejection in an allogenic heart transplantation animal model (Cooper M. et al., Transplantation. 1998 Oct. 15; 66(7):838-44). However, administration of PDTC which is a potent inhibitor for NF-κB reduced the NF-κB activity peak in the model, significantly elongating the recipient animal survival.

The normal active form of human NF-κB is a heterodimer of two DNA binding subunits, 50 kDa subunit (p50) and 65 kDa subunit (relA or p65) (Lenardo, Cell. 1989 Jul. 28; 58(2):227-9; Libermann, Mol Cell Biol. 1990 May; 10(5):2327-34; Satriano J, J Clin Invest. 1994 October; 94(4):1629-36; Neish A S et al., J Exp Med. 1992 Dec. 1; 176(6):1583-93). In a cell which is not stimulated, NF-κB binds to an inhibition molecule known as IκB and hides within the cytoplasm. After a cell is stimulated, IκB is phosphorylated and then rapidly degraded. Thereafter, NF-κB is released from IκB, thereby making it possible to translocate the transcription factor to the nucleus, in which the transcription factor binds to various DNA recognition sites to regulate gene expression (Baeurerle, 1994, supra). It has been suggested that the dissociation of the transcription factor NF-κB from the complex induces regulated transactivation of genes including interleukins (ILs)-1, -6, and -8; intracellular adhesion molecules; vascular cell adhesion molecules; and endothelial cell adhesion molecules, and plays a pivotal role in regulation of inflammatory changes (Lenardo, 1989 supra; Libermann, 1990 supra; Satriano J, 1994 supra; Neish, 1992 supra). Therefore, blockage of NF-κB may attenuate gene-mediated cardiac ischemia-reperfusion.

NF-κB may be involved in the onset of progression of tumor malignancy (Rayet B et al., Oncogene 1999 Nov. 22; 18(49)6938-47); NF-κB is involved in response of tumor cells to hypoxia stress (Royds J A et al., Mol Pathol 1998 April; 51(2):55-61); NF-κB inhibits expression of cytokines and adhesion molecules in synovial membrane cells derived from chronic rheumatoid arthritis patients (Tomita T et al., Rheumatology (Oxford) 2000 July; 39(7):749-57); suppression of coordination between a plurality of transcription factors including NF-κB changes the malignant phenotypes of various tumors (Denhardt D T, Crit Rev Oncog 1996; 7(3-4):261-91); downregulation of NF-κB activation due to green tea polyphenol blocks induction of nitric oxide synthesizing enzyme, and suppresses A431 human epidermoid carcinoma cells (Lin J K et al., Biochem Pharmacol 1999 Sep. 15; 58(6):911-5); amyloid β peptide observed in the brains of Alzheimer's disease patients binds to 75-kD neurotrophic receptor (p7.5NTR) in neuroblastoma cells to activate NF-κB in a time-dependent manner and a dose-dependent manner (Kuper P et al., J Neurosci Res 1998 Dec. 15; 54(6):798-804); TNF-α plays an important role in the onset of glomerulonephritis (Ardaillou et al., Bull Acad Natl Med 1995 January; 179(1)103-15).

A transcription factor decoy for NF-κB inhibits expression of cytokines and adhesion molecules in vivo in murine nephritis induced by TNF-α (Tomlta N et al., Gene Ther 2000 August; 7(15)1326-32); and the like.

It was suggested that NF-κB suppresses MMP1 and MMP9, members of matrix metalloproteinase (MMP), at the transcription level (Eberhardt W, Huwiler A, Beck K F, Walpen S, Pfeiischifter J. J Immunol 2000 Nov. 15, 165(10), 5788-97; M, Baker A H, Newby A C. Biochem Biophys Res Commun. Bond 1999 Oct. 22, 264(2), 561-7; Bond M, Fabunmi R P, Baker A H, Newby A C. FEBS Lett 1998 Sep. 11, 435(1), 29-34; and Kim H, Koh G. Biochem Biophys Res Commun. 2000 Mar. 16, 269(2), 401-5). MMP is a polygene family of zinc-dependent enzymes involved in degradation of extracellular matrix components.

MMP plays an important role in invasion of cancer cells by mediating degradation of extracellular matrix protein. A number of studies suggested the involvement of MMP and MMP inhibitors (TIMP) in the progression of cancer: the TIMP1 level in serum may be used as a marker for prognosis and diagnosis of colon and rectum, and a selective marker for metastatic cancer (Pellegrinl P et al., Cancer Immunol Immunother 2000 September; 49(7):388-94); expression and activity of MMP2 and MMP9 in human urinary bladder cancer cells are affected by tumor necrosis factor α and γ interferon (Shin K Y et al., Cancer Lett 2000 Oct. 31; 159(2):127-134); MMP2, MMP9 and MT1-MMP, and their inhibitors, TIMP1 and TIMP2, are expressed in ovarian epithelium tumor (Sakata K et al., Int J Oncol 2000 October; 17(4):673-681); the level of each of MMP1, MMP2, MMP3 and MMP9 and the overall MMP activity are upregulated in colon and rectum tumor, and MMP1 is most important for progression of colon and rectum cancer (Baker E A et al., Br J Surg 2000 September; 87(9):1215-1221); activated MMP2 plays an important role in invasion of urothelial cancer, and also the expression level of the activated MMP2 can be used as a useful prognosis index (Kaneda K et al., BJU Int 2000 September; 86(4):553-557); a prostaglandin synthesis inhibitor inhibits invasion of human prostate tumor cells, and reduces the release of MMP (Attiga F A et al., Cancer Res 2000 Aug. 15; 60(16):4629-37); the MMP activity of a serum euglobulin fraction increases in breast cancer and lung cancer patients, and may be used as a tumor marker for these cancers (Farias E et al., Int J Cancer 2000 Jul. 20; 89(4):389-94); a MMP inhibitor inhibits gelatin-degrading activity in tumor cells (Ikeda M et al., Clin Cancer Res 2000 August; 6(8):3290-6); induction of MMP9 due to a membrane protein LMP1 contributes to metastatic of nasopharyngeal cancer (NPC) (Horikawa T et al., Caner 2000 Aug. 15; 89(4):715-23); MMP plays an important role in an early stage of angioplasty, and a MMP inhibitor suppresses invasion and morphogenesis of human microvascular endothelial cells (Jia M C et al., Adv Exp Med Biol 2000; 476:181-94); MMP9 is expressed in invasive and recurrent pituitary adenoma and hypophysis cancer (Turner H E et al., J Clin Endocrinol Metab 2000 August; 85(8):2931-5); and the like.

MMP is also known to be involved in development of aortic aneurysm: MMP is involved in formation and rupture of cerebral aneurysm (Gaetani P et al., Neurol Res 1999 June; 21(4):385-90); a MMP-9 promotor is a risk factor for cerebral aneurysm (Peters D G et al., Stroke 1999 December; 30(12):2612-6); inhibition of MMP inhibits the growth of microaneurysm in an aneurysm model (Treharne G D et. al., Br J Surg 1999 August; 86(8):1053-8); and the like. MMP is secreted from wandering vascular smooth muscle cells, macrophage, and the like, and destroys collagen, elastin, and the like present in blood vessel walls, whereby the tension of the blood vessel is lost and the blood vessel does not resist the blood pressure and its diameter is expanded. In fact, in the blood vessel of an aneurysm, significant destruction of elastin is observed.

According to data obtained by measuring the aorta diameter of from 35-year-old to 80-year old adult males, the average was 1.5 cm to 2.0 cm. In general, the aorta having a diameter beyond 1.5 times as great as the average value is judged as an aortic aneurysm. However, according to the above-described data, one in every 400 people had an aneurysm having a diameter of 3 cm or more which is judged as aortic aneurysm. Therefore, although the degree of risk of aorta rupture is not considered here, the prevalence of aortic aneurysm is relatively high in from 35-year-old to 80-year old adult males. The prevalence is believed to be even greater in males aged 65 and above.

It has been reported that a MMP inhibitor suppresses the expansion of a blood vessel diameter in an aortic aneurysm model in a rat abdomen. A MMP inhibitor may be used in therapy for glomerulonephritis (Marti H P, Schweiz Med Wochenschr 2000 May 27; 130(21); 784-8). However, systemic administration of a MMP inhibitor causes severe side effects, and has difficulty in clinical applications for treatment (therapy and prevention) of various diseases.

Synthetic ODN as “decoy compound” cis-element blocks a nuclear factor from binding to the promoter region of its intended gene, thereby inhibiting gene transactivation of in vitro and in vivo assay systems (Sullenger, J Virol. 1991 December; 65(12):6811-6; Morishita R. et al., Contrib Nephrol. 1996; 118:254-64). Such a decoy strategy has been proposed for treatment of certain human diseases. The present inventors previously reported that transfection of E2F decoy ODN as a gene therapy model for restenosis inhibited neointimal proliferation after balloon-injury (Morishita, Proc Natl Acad Sci USA. 1995 Jun. 20; 92(13):5855-9). Recently, the present inventors succeeded in in vivo protection of myocardiac muscle from ischemic injury using a decoy for NF-κB in rats.

In the field of cardiac surgery, circulatory arrest is commonly used as a support technique in patients having aortic aneurysmal changes or in neonates having complex congenital abnormalities. However, various complications related to circulatory arrest are still unresolved, and longer duration of circulatory arrest results in a higher incidence of neurological sequelae (see Jonas R A. J. Cadiothorac Vasc Anesth. 1996; 10:66-74). During circulatory arrest, the whole body, including the brain, is ischemic, and prolonged ischemia leads to necrosis of neurons. Moreover, brain neurons, particularly neurons in the hippocampus, will die 5 to 7 days after a few minutes of ischemica, a phenomenon called delayed neuronal death (Kirino T. Brain Res. 1982; 239:57-69). Even when techniques such as deep hypothermia are used to protect the brain against ischemia injury, 45 to 60 minutes are a physical limit for maintaining circulatory arrest, and deep hypothermia is associated with various risks (increased bleeding, blood transfusion, and a decline of immunity) (see Kirklin L W, Barratt-Boyes B G. Kirklin J W, Barratt-Boyes B G, ed. Cardiac surgery. New York: Churchill Livingstone; 1993, p. 66-73). The development of better techniques for brain protection against both neuronal necrosis and delayed neuronal death resulting from ischemic disorders is desired to ameliorate the pathological conditions of surgery for aortic diseases and congenital heart diseases.

Recent studies have clarified the activation of NF-κB in neuronal damage after cerebral ischemia, indicating that NF-κB is a crucial transcription factor (see Stephenson D, Yin T, Smalstig E B, Hsu M A, Panetta J, Little S. et al., J Cereb Blood Flow Melab. 2000; 20:592-603; Schneider A, Martin-Villalba A, Weih F, Vogel J, Wirth T, Schwaninger M., Nat Med. 1999; 5:554-9; and Clemens J A, Stephenson D T, Dixon E P, Smalstig E B, Mincy R E, Rash K S et al., Brain Res Mol Brain Res. 1997; 48:187-96).

NF-κB is a transcriptional activator of a number of genes whose expression is related to ischemia-reperfusion injury (cytokines (tumor necrosis factor α (TNF-α) and interleukin 1β (IL-1β)) (see Chrisimann J W, Lancaster L H, Blackwell T S. Intensive Care Med. 1998; 24:1131-8) and adhesion molecules (intracellular adhesion molecule 1 (ICAM-1)) (Howard E F, Chen Q, Cheng C, Carroll J E, Hess-D. Neurosci Lett. 1998; 248:199-203), and the like). Further, inhibitors for NF-κB, such as aspirin, seem to block ischemic injury in neurons (Grilli M, Pizzi M, Memo M, Spano P, Science, 1996; 274:1383-5). It has been reported that transfection of decoy oligodeoxynucleotide (ODN) blocks the transcriptional activation of cytokines and adhesion molecules (Tomita N, Morishita R, Tomita S, Gibbons G H, Zhang L, Horiuchi M et al., Gene Ther. 2000; 7:1326-32). The present inventors previously reported the efficacy of transfecting NF-κB decoy ODNs to prevent ischemia-reperfusion injury in the heart (see Morishita R, Sugimoto T, Aoki M, Kida I, Tomita N, Moriguchi A et al., Nat Med. 1997; 3:894-9; and Sawa Y, Morishita R, Suzuki K, Kagisaki K, Kaneda Y, Maeda. K et al., Circulation. 1997; 96(Suppl 9):II-280-5).

Thus, it has been suggested that NF-κB is involved in various diseases via expression of a number of genes under the transcription control thereof. However, no method for effectively treating a disorder or disease associated with brain ischemia, particularly a non-invasive treatment method, has been provided. Particularly, as described above, brain ischemia is not a rare disease. As society ages, an increase in arteriosclerotic diseases inevitably leads to an increase in aortic aneurysm diseases. Considering the aging of patients, it is ideal to suppress directly the growth of aortic aneurysm using a pharmaceutical agent, however, to date such a means is not present. There is a desperate demand for development of a low-invasive therapy and prevention method for aortic aneurysm.

When the brain falls into an ischemic state due to rupture of aortic aneurysm or the like, cerebral neuropathy occurs. This disorder leads to various functional failures in nerves, potentially causing intelligence disorder, dementia, or the like. Recently, it was reported that cis element decoy oligodeoxynucleotide to NF-κB blocked gene activation mediated by ischemic injury. However, there has been substantially no low-invasive therapy or prevention method effective for treatment or prevention of disorders due to the ischemic state of the brain.

Therefore, an object of the present invention is to provide a novel protection and therapy for the brain, in which neurons are transfected with NF-κB decoy ODN to block neuronal damage after global brain ischemia. Another object of the present invention is also to test whether or not transfection of NF-κB decoy ODN to the brain through a carotid artery attenuates neuron injury after global brain ischemia in a rat model. The present inventors' object is to develop a novel pharmaceutical agent for protecting the cerebrum, which is used during global brain ischemia including circulatory arrest for cardiovascular surgery. To improve cerebral protection during circulatory arrest for cardiac surgery, the present inventors aimed to evaluate the efficacy of NF-κB decoy oligonucleotide for prevention of neuronal damage after global brain ischemia.

In another aspect, an object of the present invention is to carry out gene transfection in the brain by administrating a composition for the gene transfection through a route other than direct administration to the brain, particularly an administration route across the blood-brain barrier. This is a technique which could not be conventionally achieved. Therefore, the technical significance of the present invention is great.

### DISCLOSURE OF THE INVENTION

The present invention provides introduction of NF-κB oligodeoxynucleotide to a rat cranial nerve through a carotid artery during global brain ischemia. Polymerase chain reaction demonstrated that transfected NF-κB decoy oligodeoxynucleotide effectively inhibited expression of tumor necrosis factor α, interleukin 1β, and intracellular adhesion molecule 1 (ICAM-1) messenger RNAs one hour after global brain ischemia. Terminal deoxynucleotidyl transferase-mediated deoxyuridine nick-end labeling staining and microtubule-associated protein 2 (MAP-2) immunohistochemistry demonstrated that transfected NF-κB decoy oligodeoxynucleotide significantly attenuated neuronal damage seven days after global brain ischemia. The therapeutic transfection of NF-κB decoy oligodeoxynucleotide during brain ischemia may effectively attenuate neuronal damage, suggesting a strategy for protecting the cerebrum from global ischemia.

The present invention provides a pharmaceutical composition for treating and preventing a disease and a disorder associated with an ischemic condition of a brain, and a disease and a disorder caused by the disease and the disorder. The composition comprises at least one NF-κB decoy, and a pharmaceutically acceptable carrier. The present invention also provides a method for treating and preventing a disease and a disorder associated with an ischemic condition of a brain, and a disease and a disorder caused by the disease and the disorder. The method comprises the step of administering a composition to a subject. The composition comprises at least one NF-κB decoy, and a pharmaceutically acceptable carrier.

In one embodiment, the disease may be at least one disease selected from the group consisting of subarachnoid hemorrhage, hypertensive intracerebral hemorrhage, cerebral infarct, brain ischemia, brain tumor, head injury, chronic subdural hemorrhage, and acute subdural hemorrhage. The disease and the disorder caused by the disease and the disorder associated with the ischemic condition of the brain may be selected from the group consisting of neuropathy, motor disorders, intelligence disorder, dementia, partial paralysis, headache, and incontinence of urine. The pharmaceutically acceptable carrier may be a liposome. The NF-κB decoy may comprise a sequence GGATTTCCC. The composition may be appropriate to an administration route including a carotid artery.

According to another aspect, the present invention provides a composition for carrying out gene transfection in a brain by a route other than direct administration to the brain. The composition comprises at least one decoy, and a pharmaceutically acceptable carrier. The present invention also provides a method for carrying out gene transfection in a brain by a route other than direct administration to the brain. The method comprises the step of administering a composition into the route other than the direct administration to the brain. The composition comprises, in an appropriate form, at least one decoy, and a pharmaceutically acceptable carrier.

In one embodiment, the route other than direct administration to the brain may be an infusion to a carotid artery. In another aspect, the decoy may be NF-κB. In another aspect, the pharmaceutically acceptable carrier is a liposome.

### BRIEF DESCRIPTION OF THE DRAWINGS

FIG. 1 shows microphotographs of rat tissue one hour after reperfusion, which was transfected with FITC-labeled NF-κB decoy ODN. FITC fluorescence could be observed throughout the tissue and the nuclei of neurons in the entire brain. A shows a rat cortex section, while B shows a hippocampus section. A-1 and B-1 are photographs having a magnification factor 40, while A-2 and B-2 are photographs having a magnification factor of 100.

FIG. 2 shows graphs indicating the induction rate of mRNA in the rat hippocampus one hour after reperfusion. The levels of mRNA are normalized with respect to the mRNA level of GAPDH in each sample. The induction rate was calculated by comparing the level of a normal rat hippocampus and the level of a rat hippocampus treated by the present invention. A indicates TNF-α mRNA, B indicates IL-1β mRNA, and C indicates ICAM-1 mRNA. All values were suppressed in a NF-κB decoy group more significantly than in a S decoy group.

FIG. 3A shows photographs indicating sections across a rat hippocampus CA1 region with TUNEL staining 7 days after global brain ischemia. NF-κB decoy ODN therapy (A-1) suppressed appearance of TUNEL-positive neurons (stained in brown) better than the S decoy group (A-2). The magnification factor is 100 for both A-1 and A-2. FIG. 3B is a graph (500 μm long) showing the proportion of the TUNEL-positive neurons in the hippocampus CA1 region. The proportion of the TUNEL-positive neurons was more significantly reduced in the NF-κB decoy group than in the S decoy group (p<0.01).

FIG. 4A shows photographs indicating sections across a rat hippocampus CA1 region with MAP2 immunological staining 7 days after global brain ischemia. NF-κB decoy ODN therapy (A-1) suppressed appearance of MAP2-positive neurons (no immune response in cytosol) better than the S decoy group (A-2). The magnification factor is 100 for both A-1 and A-2. FIG. 4B is a graph showing the proportion of the MAP2-positive neurons in the hippocampus CA1 region (500 μm in length). The proportion of the MAP2-positive neurons was more significantly maintained in the NF-κB decoy group than in the S decoy group (p<0.01).

### BEST MODE FOR CARRYING OUT THE INVENTION

Hereinafter, the present invention will be described. It should be understood throughout the present specification that articles for a singular form (e.g., “a”, “an”, “the”, etc. in English; “ein”, “der”, “das”, “die”, etc. and their inflections in German; “un”, “une”, “le”, “la”, etc. in French; “un”, “una”, “el”, “la”, etc. in Spanish, and articles, adjectives, etc. in other languages) include the concept of their plurality unless otherwise mentioned. It should be also understood that the terms as used herein have definitions typically used in the art unless otherwise mentioned.

The term “decoy” or “decoy compound” refers to a compound which binds to a site on a chromosome, which a transcription factor, such as NF-κB and the like, binds to, or a site on a chromosome, which another transcriptional regulatory factor for a gene controlled by a transcription factor, such as NF-κB and the like (hereinafter referred to as a target binding site) to antagonize the binding of NF-κB or other transcription factors to these target binding sites. Representatively, the decoy or the decoy compound includes a nucleic acid and analogs thereof.

When a decoy is present within a nucleus, the decoy conflicts with a transcriptional regulatory factor competing for a target binding site for the transcriptional regulatory factor. As a result, a biological function which would be generated by binding of the transcriptional regulatory factor to the target binding site is inhibited. The decoy contains at least one nucleic acid sequence capable of binding to a target binding sequence. A decoy can be used for preparation of a pharmaceutical composition according to the present invention as long as the decoy has activity to bind to a target binding sequence.

Examples of the decoy include oligonucleotides containing GGGATTTC concerning NF-κB. Preferable examples of the decoy include 5′-CCTTGAAGGGATTTCCCTCC-3′ (SEQ ID NO: 1) (NF-κB decoy), 5′-GATCTAGGGATTTCCGGGAAATGAAGCT-3′ (SEQ ID NO: 2) (STAT-1 decoy), 5′-AGCTTGAGATAGAGCT-3′ (SEQ ID NO: 3) (GATA-3 decoy), 5′-GATCAAGACCTTTTCCCAAGAAATCTAT-3′ (SEQ ID NO: 4) (STAT-6 decoy), 5′-AGCTTGTGAGTCAGAAGCT-3′ (SEQ ID NO: 5) (AP-1 decoy), and 5′-AATTCACCGGAAGTATTCGA-3′ (SEQ ID NO: 6) (Ets decoy), 5′-TGACGTCA-3′ (CRE decoy sequence) or oligonucleotide containing complements thereof, mutants thereof, or compounds containing these molecules therein. The oligonucleotides may be either DNA or RNA. The oligonucleotides may also include a modified nucleic acid and/or pseudonucleic acid therein. Further, these oligonucleotides may be mutants thereof, or compounds containing them therein. The oligonucleotides may have a single strand or double strands, or may be linear or circular. The mutants are nucleic acids having the above-described sequences, a part of which has a mutation, a substitution, an insertion, or a deletion, and which specifically antagonize a transcription factor, such as NF-κB and the like, or another transcriptional regulatory factor for a gene controlled by a transcription factor, such as NF-κB and the like, with respect to the nucleic acid binding site to which the factor binds. More preferable examples of the decoy for the transcription factor, such as NF-κB and the like, or the other transcriptional regulatory factor for a gene controlled by a transcription factor, such as NF-κB and the like, include double-strand oligonucleotides containing one or a plurality of the above-described nucleic acid sequences, or mutants thereof. Nucleic acids containing one or a plurality of the above-described nucleic acid sequences are called double decoy when the number of nucleic acid sequences contained is two or triple decoy when the number of nucleic acid sequences contained is three, indicating the number of nucleic acid sequences.

The oligonucleotides for use in the present invention include oligonucleotides modified so as to resist in vivo degradation, and the like, such as oligonucleotides (S-oligo) having a thiophosphatediester bond which is a phosphatediester bond whose oxygen atom is replaced with a sulfur atom, oligonucleotides whose phosphatediester bond is substituted with a methylphosphate group having no electronic charge, and the like.

The decoy of the present invention can be produced with chemical or biochemical synthesis methods known in the art. For example, when a nucleic acid is used as a decoy compound, nucleic acid synthesis methods commonly used in genetic engineering can be employed. For example, a DNA synthesizer may be used to directly synthesize intended decoy nucleic acids. Further, these nucleic acids, nucleic acids containing the nucleic acids, or parts thereof may be synthesized, followed by amplification using a PCR method, a cloning vector, and the like. Furthermore, nucleic acids obtained by these methods are cleaved using a restriction enzyme, or the like, and linked or the like using DNA ligase, or the like to produce an intended nucleic acid. To obtain decoy nucleic acids which are more stable in cells, base, sugar and phosphate portions of the nucleic acids may be subjected to chemical modification, such as alkylation, acylation, or the like.

The present invention provides a pharmaceutical composition comprising the above-described decoy compound alone or in combination with a stabilizing compound, a diluent, a carrier or another component, or a pharmaceutical agent.

The pharmaceutical composition of the present invention may be used in such a form that the decoy is taken into cells in an affected part or cells in an intended tissue.

The pharmaceutical composition of the present invention is administered in any aseptic biocompatible pharmaceutical carrier (including, but not limited to, physiological saline, buffered physiological saline, dextrose, and water). A pharmaceutical composition of any of these molecules mixed with an appropriate excipient, an adjuvant, and/or a pharmaceutically acceptable carrier may be administered to patients alone or in combination with another pharmaceutical agent in a pharmaceutical composition. In an embodiment of the present invention, the pharmaceutically acceptable carrier is pharmaceutically inactive.

The administration of the pharmaceutical composition of the present invention is achieved orally or parenterally. Parenteral delivery methods include topical, intra-arterial (e.g., through a carotid artery), intramuscular, subcutaneous, intramedullary, into subarachnoid space, intraventricular, intravenous, intraperitoneal, or intranasal administrations. In the present invention, any route may be possible as long as the composition is delivered through the route to a site to be treated, i.e., brain. The present inventors demonstrated that the present invention can be applied to, for example, infusion from a cervical part which requires passing across the blood-brain barrier. Thus, the present invention provides such an advantageous effect which could not be achieved by conventional techniques. Therefore, in a preferred embodiment of the present invention, routes which have to pass across the blood-brain barrier (e.g., oral administration, and parenteral administration (e.g., administration from cervical parts)). More preferably, the administration route may be the infusion from cervical parts (e.g., through a carotid artery). Therefore, the present invention provides a novel treatment method for carrying out gene transfection to the brain using a route through a carotid artery, and a composition for use in the method.

In addition to the decoy compound, these pharmaceutical compositions contain an appropriate pharmaceutically acceptable carrier, including another compound for accelerating the processing of the decoy compound so as to produce an excipient or a preparation which can be pharmaceutically used. Further details of techniques for preparation and administration of the decoy compound are described in, for example, the latest version of Japanese Pharmacopoeia with the latest supplement, and “REMINGTON'S PHARMACEUTICAL SCIENCES” (Maack Publishing Co., Easton, Pa.).

A pharmaceutical composition for oral administration may be prepared using a pharmaceutically acceptable carrier well known in the art in an administration form suitable for administration. Such a carrier can be prepared as a tablet, a pill, a sugar-coated agent, a capsule, a liquid, a gel, a syrup, a slurry, a suspension, or the like, which is suited for the patient to take the pharmaceutical composition.

The pharmaceutical composition for oral use may be obtained in the following manner: an active compound is combined with a solid excipient, the resultant mixture is pulverized if necessary, an appropriate compound is further added if necessary to obtain a tablet or the core of a sugar-coated agent, and the granular mixture is processed. The appropriate excipient may be a carbohydrate or protein filler, including, but not being limited to, the following: sugar including lactose, sucrose, mannitol, or sorbitol; starch derived from maize, wheat, rice, potato, or other plants; cellulose such as methylcellulose, hydroxypropylmethylcellulose, or sodium carboxymethylcellulose; and gum including gum Arabic and gum tragacanth; and protein such as gelatin and collagen. A disintegrant or a solubilizing agent such as crosslinked polyvinyl pyrrolidone, agar, alginic acid or a salt thereof (e.g., sodium alginate) may be used if necessary.

The sugar-coated agent core is provided along with an appropriate coating, such as a condensed sugar solution. The sugar-coated agent core may also contain gum arabic, talc, polyvinyl pyrrolidone, carbopolygel, polyethylene glycol, and/or titanium dioxide, a lacquer solution, and an appropriate organic solvent or a solvent mixed solution. To identify a product, or characterize the amount of an active compound (i.e., dose), dye or pigment may be added to tablets or sugar-coated agents.

The pharmaceutical preparation which may be orally used may contain, for example, a soft sealed capsule consisting of a gelatin capsule, gelatin and coating (e.g., glycerol or sorbitol). The gelatin capsule may contain an active component mixed with a filler or binder such as lactose or starch, a lubricant such as talc or magnesium stearate, and optionally a stabilizer. In the soft capsule, the decoy compound may be dissolved or suspended in an appropriate liquid, such as fatty oil, liquid paraffin or liquid polyethylene glycol, with or without a stabilizer.

The pharmaceutical preparation for parenteral administration contains an aqueous solution of an active compound. For the purpose of injection, the pharmaceutical composition of the present invention is prepared in an aqueous solution, preferably Hank's solution, Ringer's solution, or a physiologically suitable buffer such as a buffered physiological saline. The aqueous suspension for injection may contain a substance for increasing the viscosity of a suspension (e.g., sodium carboxymethylcellulose, sorbitol, or dextran). Further, the suspension of the active compound may be prepared as an appropriate oily suspension. Appropriate lipophilic solvents or vehicles include fatty acid such as sesame oil, synthetic fatty acid ester such as ethyl oleate or triglyceride, or liposome. The suspension may contain a stabilizer which allows a high-concentration solution preparation, or an appropriate pharmaceutical agent or reagent for increasing the solubility of the compound, if necessary.

For topical or intranasal administration, an appropriate penetrant for the specific barrier to be penetrated may be used in the preparation. Such a penetrant is generally known in the art.

The pharmaceutical composition of the present invention may be produced using a method similar to method known in the art (e.g., conventional mixing, dissolution, rendering to granules, preparation of a sugar-coated agent, elutriation, emulsification, capsulation, inclusion, or freeze drying).

Preferably, in the case of parenteral administration, such as topical administration or infusion from a cervical portion to cell of an affected part or cells of an intended tissue, the pharmaceutical composition of the present invention may contain a synthetic or naturally-occurring hydrophilic polymer as a carrier. Examples of such a hydrophilic polymer include hydroxypropylcellulose and polyethylene glycol. The decoy compound of the present invention may be mixed with the above-described hydrophilic polymer in an appropriate solvent. The solvent may be removed by a method such as air drying. The resultant compound may be shaped into a desired form, such as sheet, and then may be given to a target site. Such a preparation containing a hydrophilic polymer has a small moisture content, and an excellent shelf life, and an excellent retentivity of the decoy compound since the preparation absorbs water to be turned into gel when used.

Alternatively, when a nucleic acid or a modification thereof is employed as a decoy, the pharmaceutical composition of the present invention is advantageously used in a form which is generally used in gene introduction methods, such as a membrane fusion liposome preparation using Sendai virus (HVJ) or the like, a liposome preparation using endocytosis or the like, a preparation containing a cationic lipid such as Lipofectamine (Gibco BRL) or the like, or a viral preparation using a retrovirus vector, an adenovirus vector, or the like. Particularly, a membrane fusion liposome preparation is preferable.

The liposome preparation is any of the liposome constructs which are a large unilamellar vesicle (LUV), a multilammelar vesicle (MLV), and a small unilamellar vesicle (SUV). The LUV has a particle system ranging from about 200 to about 1000 nm. The MLV has a particle system ranging from about 400 to about 3500 nm. The SUV has a particle system ranging from about 20 to about 50 nm. The membrane fusion liposome preparation using HVJ or the like preferably employs MLV having a particle system ranging from 200 nm to 1000 nm.

There is no particular limitation on a method for producing liposomes as long as the liposomes hold a decoy. The liposomes can be produced by a commonly used method, such as, for example, a reversed phase evaporation method (Szoka, F et al., Biochim. Biophys. Acta, Vol. 601 559(1980)), an ether infusion method (Deamer, D. W.: Ann. N.Y. Acad. Sci., Vol. 308-250(1978)), a surfactant method (Brunner, J et al.: Biochim. Biophys. Acta, Vol. 455 322(1976)), or the like.

Examples of lipids for forming a structure of a liposome include phospholipids, cholesterols, nitrogen lipids, and the like. Generally, phospholipids are preferable, including naturally-occurring phospholipids, such as phosphatidylcholine, phosphatidylserine, phosphatidylglycerol, phosphatidylinositol, phosphatidylethanolamine, phosphatidic acid, cardiolipin, sphingomyelin, egg yolk lecithin, soybean lecithin, lysolecithin, and the like, or the corresponding phospholipids hydrogenated by a commonly used method, and in addition, synthetic phospholipids, such as dicetylphosphate, distearoylphosphatidylcholine, dipalmitoylphosphatidylcholine, dipalmitoylphosphatidylethanolamine, dipalmitoylphosphatidylserine, eleostearoylphosphatidylcholine, eleostearoylphosphatidylethanolamine, eleostearoylphosphatidylserine, and the like.

The lipids including these phospholipids can be used alone or with at least two in a combination. In this case, lipids having an atom group having a positive group, such as ethanolamine, choline, or the like, within the molecule can be used to increase the binding rate of an electrically negative decoy nucleic acid. In addition to the major phospholipids used to form liposomes, an additive, such as cholesterols stearylamine, α-tocopherol, or the like, which are generally known as an additive for formation of liposomes, can be used.

The thus-obtained liposomes can additionally contain a substance for promoting membrane fusion, such as a membrane fusion promoting protein purified from HVJ, inactivated HVJ, Sendai virus, or the like, so as to accelerate uptake into cells at an affected site or cells in an intended tissue.

An exemplary method for producing a liposome preparation will be specifically described below. For example, the above-described substance for forming a liposome is dissolved along with cholesterol in an organic solvent, such as tetrahydrofuran, chloroform, ethanol, or the like. The resultant solution is put into an appropriate vessel, followed by removal of the solvent under reduced pressure, thereby forming a film of the liposome forming substance on an inside wall of the vessel. A buffer solution containing a decoy is added to the vessel followed by agitation. The above-described membrane fusion promoting substance is added to the resultant liposome if necessary, followed by isolation of the liposome. The thus-obtained liposome containing the decoy can be suspended in an appropriate solvent or can be freeze-dried and thereafter dispersed in an appropriate solvent. The resultant suspension can be used in treatment. The membrane fusion promoting substance may be added in the interim period after the isolation of the liposome and before use.

The composition or kit of the present invention may further comprise a biocompatible material. Such a biocompatible material may contain at least one selected from the group consisting of silicone, collagen, gelatin, glycolic acid-lactic acid copolymers, ethylene-vinyl acetate copolymers, polyurethane, polyethylene, polytetrafluoroethylene, polypropylene, polyacrylate, and polymethacrylate, for example. Silicone is preferable because of its ease of molding. Examples of biodegradable polymers include collagen; gelatin; polymers or copolymers synthesized by dehydration polycondensation without a catalyst from at least one selected from the group consisting of α-hydroxycarboxylic acids (e.g., glycolic acid, lactic acid, hydroxybutyric acid, and the like), hydroxydicarboxylic acids (e.g., malic acid and the like) and hydroxytricarboxylic acids (e.g., citric acid and the like), or a mixture thereof; poly-α-cyanoacrylate ester; polyamino acids (e.g., poly-γ-benzil-L-glutamic acid and the like), polymerizable acid anhydrides of maleic anhydride-based copolymers (e.g., styrene-maleic acid copolymers and the like); and the like. The type of the polymerization is any of random, block, and graft. When α-hydroxycarboxylic acids, hydroxydicarboxylic acids, and hydroxytricarboxylic acids have the center of optical activity in the molecule, any of D-isomers, L-isomers, and DL-isomers can be used. Preferably, a glycolic acid-lactic acid copolymer may be used.

In one embodiment, the composition or kit of the present invention may be provided in a form of sustained release. The sustained-release dosage form may be any known form in the art as long as it is used in the present invention. Examples of such a form include rod forms (pellet forms, cylinder forms, needle forms, and the like), tablet forms, disk forms, ball forms, and sheet forms. Methods for preparing a sustained-release form are known in the art and disclosed in, for example, Japanese Pharmacopoeia, U.S. Pharmacopoeia, other countries' Pharmacopoeias, and the like. Examples of methods for producing a sustained-release preparation (prolonged-administration preparation) include a method of utilizing disaggregation of a drug from a complex, a method of using an aqueous suspension for injection, a method of using an oil solution for injection or an oil suspension for injection, a method of using an emulsion for injection (o/w type and w/o type emulsions for injection, and the like), and the like.

In the case of the sustained-release form, a sustained-release preparation (mini-pellet preparation or the like) can be embedded in the vicinity of a site to which the preparation is to be administered. Alternatively, an osmotic pump or the like can be used to administer the sustained-release preparation continuously and gradually.

Injection agents can be prepared by a method well known in the art. For example, a component is dissolved in an appropriate solvent (physiological saline, a buffer solution such as PBS, sterilized water, or the like), followed by filter sterilization using a filter or the like. Thereafter, an aseptic vessel (e.g., ampoule or the like) is filled with the resultant solution, thereby preparing the injection agent. The injection agents may contain a commonly used pharmaceutical carrier if necessary. In the case of the liposome form, a reagent required for liposome preparations, such as suspension agents, cryogen, and cryogen condensed by centrifugation, can be added. The liposome is preferably administered parenterally. Therefore, when the liposome is administered, a non-invasive catheter, a non-invasive syringe, or the like can be used for the administration. As an administration method using a non-invasive catheter, the composition of the present invention is infused directly into brain or through a carotid artery, for example.

The pharmaceutical composition of the present invention includes a composition containing an effective amount of decoy compound which can achieve the intended purpose of the decoy compound. “Therapeutically effective amount” or “pharmacologically effective amount” are terms which are well recognized by those skilled in the art and which refer to an amount of pharmaceutical agent effective for production of an intended pharmacological effect. Therefore, the therapeutically effective amount is an amount sufficient for reducing the manifestation of diseases to be treated. A useful assay for confirming an effective amount (e.g., a therapeutically effective amount) for a predetermined application is to measure the degree of recovery from a target disease. An amount actually administered depends on an individual to be treated. The amount is preferably optimized so as to achieve a desired effect without a significant side effect. The determination of the therapeutically effective dose is within the ability of those skilled in the art.

A therapeutically effective dose of any compound can be initially estimated using either a cell culture assay or any appropriate animal model. The animal model is used to achieve a desired concentration range and an administration route. Thereafter, such information can be used to determine a dose and route useful for administration into humans.

The therapeutically effective amount refers to an amount of a decoy compound which results in amelioration of symptoms or conditions of a disease. The therapeutic effect and toxicity of such a compound may be determined by standard pharmaceutical procedures in cell cultures or experimental animals (e.g., ED₅₀, a dose therapeutically effective for 50% of a population; and LD₅₀, a dose lethal to 50% of a population). The dose ratio between therapeutic and toxic effects is a therapeutic index, and it can be expressed as the ratio of ED₅₀/LD₅₀. Pharmaceutical compositions which exhibit high therapeutic indices are preferable. The data obtained from cell culture assays and animal studies can be used in formulating a range of amount for use in humans. The dosage of such compounds lies preferably within a range of circulating concentrations that include the ED₅₀ with little or no toxicity. Such a dosage may vary within this range depending upon the dosage form employed, the susceptibility of a patient, and the route of administration. As an example, the dose of a decoy is appropriately selected depending on the age and other conditions of a patient, the type of a disease, the type of the decoy employed, and the like. For example, the decoy is administered to brain once to several times per day in an amount of 10 to 10,000 nmole per time. The decoy is administered to a carotid artery, generally, once to several times per day in an amount of 10 to 10,000 nmole per time.

The exact dose is chosen by an individual physician in view of the condition of a patient to be treated. Doses and administration are adjusted to provide a sufficient level of the active portion, or to hold a desired effect. Additional factors to be considered include the severity of the condition of a disease (e.g., the size and location of a tumor; the age, weight and sex of a patient; a diet-limiting time and frequency of administration, a combination of drugs, reaction susceptibility, and resistance/response to treatment). A sustained action pharmaceutical composition may be administered every 3 to 4 days, every week, or once per two weeks, depending on the half life and clearance rate of a specific preparation. Guidance for specific doses and delivery methods are provided in publications known in the art.

Medicaments containing the thus-obtained decoy as a major component can be administered in various manners, depending on the type of disease, the type of the decoy employed, and the like. For example, the medicament can be intravascularly administered, applied to the site of a disease, administered to the disease site, or intravascularly administered to the disease site, for ischemic diseases, inflammatory diseases, autoimmune diseases, and cancer metastasis and invasion, and cachexia. More specifically, for example, when PTCA is performed for infarct of an organ, the medicament can be administered into a blood vessel of an affected part at the same time or before or after the PTCA. In organ transplantation or the like, an organ to be transplanted may be treated in advance with a preparation for use in the present invention. Further, for example, the medicament can be infused directly to a joint in the case of chronic articular rheumatism or the like. For example, the medicament may be infused directly to the brain.

Disorders or diseases targeted by the compound of the present invention are attributed to shortage of blood in the brain due to rupture of a blood vessel in the brain, or the like. Such disorders or diseases herein refer to diseases in connection with the ischemic condition of the brain. Examples of such disorders or diseases include stroke (e.g., subarachnoid hemorrhage, transient brain ischemia, and cerebral arteriosclerosis), hypertensive intracerebral hemorrhage, cerebral infarct, brain ischemia, rupture of a blood vessel due to brain tumor, head injury, chronic and acute subdural hemorrhage, cerebrovascular occlusion, cerebral thrombosis, cerebral hemorrhage, cerebrovascular moyamoya disease (Moya Moya disease), cerebrovascular dementia, Alzheimer type dementia, a sequelae of cerebral hemorrhage, a sequelae of cerebral infarct, and the like. The present invention is also effective for treatment and prevention of disorders or sequelae (e.g., neuropathy and the like) caused by diseases in connection with an ischemic state of the brain.

“Subarachnoid hemorrhage” refers to a condition in which hemorrhage occurs in subarachnoid space. Except for subarachnoid hemorrhage due to head injury, the most frequent cause is rupture of cerebral aneurysm (60 to 80%). Other leading causes include cerebral arteriovenous malformation rupture (10%), hypertensive intracerebral hemorrhage (10%), and others. In the case of hypertensive intracerebral hemorrhage, it is believed that hemorrhage ruptures the ventricle and blood flows into the subarachnoid space. Chronic cerebrospinal fluid circulation disorders due to subarachnoid space occlusion occur at about 10%, possibly resulting in normal pressure hydrocephalus. Conventionally, the resultant symptoms, such as dysbasia, incontinence of urine, and intelligence disorders, are recovered by cerebrospinal fluid shunt, but about 30% of the patients die before hospitalization. Other conventional therapies include a method of pinching an aneurysm with a clip of titanium to prevent re-hemorrhage, a method of inserting a thin tube into an artery of a thigh and filling an aneurysm with a coil of titanium, and the like. Thus, in many cases, subarachnoid hemorrhage is surgically treated, and is not fundamentally solved. The present invention may be effective for treatment and prevention of all of the above-described subarachnoid hemorrhages.

Hypertensive intracerebral hemorrhage refers to a condition in which fibrinoid necrosis occurs in the wall of a small artery in the brain due to hypertension of long duration, and the wall is ruptured, resulting in hemorrhage. Hypertensive intracerebral hemorrhage occupies 20% of cerebrovascular disorders. It is also believed that hypertensive intracerebral hemorrhage occurs because micro cerebral aneurysm occurs and ruptures. A high incidence occurs in people in their 60s. The occurrence site of the hemorrhage is the cerebral basal ganglia thalamus (60%), under cerebral cortex (20%), cerebellum (10%), and mesencephalon pons (10%). Conventionally, conservative therapy is performed, including prevention of extension of the hemorrhage, reduction of intracranial pressure, prevention of systemic complications, and early rehabilitation. The main purpose of surgical therapy is lifesaving. There was a report that long-term therapy results had no difference between cases with and without surgery. Therefore, there has been a demand for an effective method for treatment and prevention as an alternative to surgery. The present invention provides an effective treatment and prevention of all hypertensive intracerebral hemorrhages.

Brain infarct refers to a condition in which a blood vessel is completely occluded, leading to the death of a part of the brain. Brain ischemia refers to a condition in which a blood vessel is narrowed and therefore a sufficient amount of blood is not supplied to the brain. It is said that unless at least 20 ml of blood per minute per 100 g of the brain is supplied to the brain, function of the brain is impaired. Symptoms due to cerebral infarct include partial paralysis and sensory disorders. Such symptoms are very likely to occur in the early morning, particularly when a disorder, such as cardiac arrhythmia or the like, is present. The current most commonly used method is to perform thrombolysis as early as possible after a blood vessel is occluded (preferably, within three hours after the onset of thrombus). This is because if at least three hours passes after the onset of the symptom, thrombolysis may cause hemorrhage (this condition is called hemorrhagic infarct, which is an extremely serious condition). Recently, MRI or DWI achieves early detection of infarct. To date, however, there is substantially no fundamental therapy for brain ischemia. The present invention may be effective for treatment and prevention of the brain ischemia and cerebral infarct.

Brain tumor as used herein refers to tumor which occurs within the skull, including primary or metastatic neoplasm developed from not only the brain but also tissue present within the skull (e.g., bones, meninges, blood vessels, hypophysis, cranial nerves, congenital retained tissue, and the like). Granulomas due to parasites, tuberculosis, or the like may be included in the brain tumor. The brain tumor is typically divided into categories in accordance with the WHO's internationally unified system, including glioma, meningioma, pituitary adenoma, schwannoma, and the like. The brain tumor is basically treated by removing the tumor by surgery. When radical surgery is difficult due to the site at which the tumor is located, radiation therapy, chemotherapy, or immunotherapy is used. Therefore, treatment using the decoy of the present invention provides a method for effective therapy and prevention having a novel aspect.

Head injury refers to any damage which is generated by external force acting on the head. The head injury is divided, according to the time of the development of the injury, into three phases: an acute phase (within three days after trauma); a subacute phase (from about 4 to about 20 days after trauma); and a chronic phase (at least 3 weeks after trauma). This categorization is involved in prognosis. The head injury is generated by bruising the head due to traffic accidents, fall, collapse, or the like. The head injury ranges from no observed abnormality to various conditions associated with brain contusion or intracerebral hematoma. The head injury is divided in various manners, generally into open and closed head injuries, and further scalp, cranial bone, intracranial head injuries, depending on a site. The present invention may be effective for treatment and prevention of rupture of cerebral blood vessels due to all of these head injuries.

Subdural hemorrhage refers to flowable hematoma which has a coating layer formed between a dura and a surface of the brain. The subdural hemorrhage is divided into acute and chronic subdural hemorrhages. The acute subdural hemorrhage is developed when a pontine vein is extended and ruptured due to displacement between the brain and the cranial bone caused by trauma, or when hemorrhage due to brain contusion caused by trauma extends to subdural space. The chronic subdural hemorrhage refers to a condition in which a relatively small amount of subdural hemorrhage caused by trauma gradually increases over several weeks to several months, resulting in lowered consciousness, psychotic manifestation, or motor paralysis. A fibrous coating layer is gradually formed around subdural hemorrhage as a result of biological reactions. Since such a coating layer is semipermeable, surrounding cerebrospinal fluid components are gradually drawn into the subdural hemorrhage which is in turn enlarged. As a result, a symptom, such as partial paralysis, headache, or the like, may be initially developed and then a typical symptom, such as dementia, dysbasia, incontinence of urine, or the like may be developed. The subdural hemorrhage is treated by a method of removing blood by surgery with local anesthesia; ventricle-abdominal cavity shunt which provides communication between the ventricle and the abdomen using a tube; or the like. The present invention may be effective for treatment and prevention of all of these subdural hemorrhages.

Therefore, diseases and disorders caused by diseases and disorders associated with an ischemic state of the brain are herein selected from the group consisting of neuropathy, motor disorders, intelligence disorder, dementia, partial paralysis, headache, and incontinence of urine.

Sites to be treated by the present invention may be derived from any type of organism. Organisms to be treated by the present invention include vertebrates and invertebrates, preferably mammals (e.g., primates, rodents, and the like), more preferably primates, and most preferably humans.

The composition and kit of the present invention are used typically with supervision of a physician, or without it when permitted by an authority and a law of a concerned country.

In another aspect, the present invention provides a kit for treating ischemic brain disorders. This kit comprises the decoy or the decoy compound of the present invention; and a manufacturer's instruction which provides guidelines for administration of the decoy or the decoy compound. The manufacturer's instruction describes a statement indicating an appropriate method for administrating the decoy or the decoy compound. The manufacturer's instruction is prepared in accordance with a format defined by an authority of a country in which the present invention is practiced (e.g., Health, Labor and Welfare Ministry in Japan, Food and Drug Administration (FDA) in U.S., and the like), explicitly describing that the instruction is approved by the authority. The manufacturer's instruction is a so-called package insert, and typically provided in a medium including, but not limited to, paper media, electronic media (e.g., web sites and electronic mails provided on the Internet).

The amount of the decoy or decoy compound of the present invention can be easily determined by those skilled in the art with reference to the purpose of use, a target disease (type, severity, and the like), the patient's age, weight, sex, and case history, the form or type of a biologically active substance in cells, the form or type of the cells, and the like.

The frequency of the method of the present invention which is applied to a subject (patient) is also determined by the those skilled in the art with respect to the purpose of use, a target disease (type, severity, and the like), the patient's age, weight, sex, and case history, the progression of the therapy, and the like. Examples of the frequency include once per day to several months (e.g., once per week to once per month). Preferably, administration is performed once per week to month with reference to the progression.

In another embodiment, the treatment method of the present invention may further comprise administrating another pharmaceutical agent. Such a pharmaceutical agent may be any medicament in the art, including any pharmaceutical agents (e.g., antibiotics and the like) known in the pharmacology field. Of course, such a pharmaceutical agent may be at least two other pharmaceutical agents. Examples of the pharmaceutical agents include those described in the latest Japanese Pharmacopoeia, the latest U.S. Pharmacopoeia, the latest Pharmacopoeias in other countries, and the like. The pharmaceutical agents may be those which preferably have an effect on cerebral ischemic diseases (e.g., antiplatelets, cranial nerve function improvers, cerebral metabolism improvers, bloodstream improver, and the like).

In a preferred embodiment, the decoy or decoy compound of the present invention may be present in an amount of at least 0.1 ng/ml, and more preferably 1.0 ng/ml. In another preferred embodiment, the decoy or decoy compound of the present invention may be present in an amount of at least 2.0 ng/ml, at least 5.0 ng/ml, at least 10.0 ng/ml, at least 20.0 ng/ml, at least 50.0 ng/ml, at least 100.0 ng/ml, at least 200.0 ng/ml, at least 500.0 ng/ml, at least 1.0 μg/ml, at least 2.0 μg/ml, at least 5.0 μg/ml, at least 10.0 μg/ml, at least 100.0 μg/ml, or at least 1 mg/ml.

Molecular biological techniques, biochemical techniques, and microbiological techniques used herein are well known and commonly used in the art, and described in, for example, Ausubel F. A. et al. ed (1988), Current Protocols in Molecular Biology, Wiley, New York, N.Y.; Sambrook J et al. (1987) Molecular Cloning: A Laboratory Manual, 2nd Ed., Cold Spring Harbor Laboratory Press, Cold Spring Harbor, N.Y.; Jikken-Igaku “Idenshi-Donyu & Hatsugen-Kaiseki-Jikkenho [Experimental medicine “Experimental methods for Gene introduction & Expression Analysis”, Yodo-sha, special issue, 1997; and the like.

As used herein, “nucleic acid”, “nucleic acid molecule”, “polynucleotide”, and “oligonucleotide” are herein interchangeably used to refer to a macromolecule (polymer) comprising a series of nucleotides, unless otherwise specified. A nucleotide refers to a nucleoside whose base is a phosphoric ester. The base of the nucleotide is a pyrimidine or purine base (pyrimidine nucleotide and purine nucleotide). Polynucleotides include DNA or RNA.

Further, sequences obtained by homology search through a genetic information database, such as GenBank (genome data by the human genome project) using software, such as BLAST, based on the sequence of the decoy of the present invention, also fall within the scope of the present invention.

Comparison of the identity of base sequences is herein calculated using BLAST (sequence analyzing tool) with default parameters.

As used herein, “polynucleotides hybridizing under stringent conditions” refer to conditions commonly used and well known in the art. Such a polypeptide can be obtained by conducting colony hybridization, plaque hybridization, Southern blot hybridization, or the like using a polynucleotide selected from the polynucleotides of the present invention. Specifically, a filter on which DNA derived from a colony or plaque is immobilized is used to conduct hybridization at 65° C. in the presence of 0.7 to 1.0 M NaCl. Thereafter, a 0.1 to 2-fold concentration SSC (saline-sodium citrate) solution (1-fold concentration SSC solution is composed of 150 mM sodium chloride and 15 mM sodium citrate) is used to wash the filter at 65° C. Plynucleotide identified by this method is referred to as “polynucleotides hybridizing under stringent conditions”. Hybridization can be conducted in accordance with a method described in, for example, Molecular Cloning 2nd ed., Current Protocols in Molecular Biology, Supplement 1-38, DNA Cloning 1: Core Techniques, A Practical Approach, Second Edition, Oxford University Press (1995), and the like. Here, sequences hybridizing under stringent conditions exclude, preferably, sequences containing only A or T.

“Homology” of genes refers to the degree of the identity between two or more gene sequences. Therefore, the greater the homology between certain two genes, the greater the identity or similarity between their sequences. Whether or not two genes have homology, can be studied by comparing two sequences directly or by hybridization under stringent conditions. When two gene sequences are directly compared to each other, the genes have homology if representatively at least 50%, preferably at least 70%, more preferably at least 80%, 90%, 95%, 96%, 97%, 98%, or 99% of the DNA sequence of the genes are identical.

As used herein, “fragment” of a nucleic acid molecule refers to a polynucleotide having a length which is shorter than the full length of the reference nucleic acid molecule but sufficient for use at least as a factor in the present invention. Therefore, the fragment as used herein refers to a polynucleotide having a sequence length ranging from 1 to n−1 with respect to the full length of the reference polynucleotide (the length is n). The length of the fragment can be appropriately changed depending on the purpose. For example, in the case of polynucleotides, the lower limit of the length of the fragment includes 5, 6, 7, 8, 9, 10, 15, 20, 25, 30, 40, 50, 75, 100 and more nucleotides. Lengths represented by integers which are not herein specified (e.g., 11 and the like) may be appropriate as a lower limit.

“Hybridizable polynucleotide” refers to a polynucleotide which can hybridize other polynucleotides under the above-described hybridization conditions. Specifically, the hybridizable polynucleotide includes at least a polynucleotide having a homology of at least 60% to the base sequence of SEQ ID NO: 1, preferably a polynucleotide having a homology of at least 80%, and more preferably a polynucleotide having a homology of at least 95%. Homology as described herein is represented by a score using the search program BLAST which employs an algorithm developed by Altschul et al. (J. Mol. Biol. 215, 403-410(1990)), for example.

“Derived oligonucleotide” refers to an oligonucleotide including a derivative of a nucleotide or having a linkage between nucleotides which is not normal. Specifically, examples of such an oligonucleotide include a derived oligonucleotide in which a phosphodiester bond is converted to a phosphothioate bond, a derived oligonucleotide in which phosphodiester bond is converted to N3′-P5′ phosphoramidate bond, a derived oligonucleotide in which ribose and phosphodiester bond are converted to peptide-nucleic acid bond, a derived oligonucleotide in which uracil is substituted with C-5 propynyl uracil, a derived oligonucleotide in which uracil is substituted with C-5 thiazole uracil, a derived oligonucleotide in which cytosine is substituted with C-5 propynyl cytosine, a derived oligonucleotide in which cytosine is substituted with phenoxazine-modified cytosine, a derived oligonucleotide in which ribose is substituted with 2′-O-propynyl ribose, a derived oligonucleotide in which ribose is substituted with 2′-methoxyethoxy ribose, and the like.

As used herein, “biological activity” refers to the activity which a certain factor (e.g., polynucleotide or polypeptide) has within an organism, including activity exhibiting various functions. For example, when the certain factor is a transcription factor, its biological activity includes activity to regulate transcriptional activity. When the certain factor is an enzyme, its biological activity includes enzymatic activity. As another example, when the certain factor is a ligand, its biological activity includes binding to a receptor to which the ligand corresponds. In one embodiment of the present invention, its biological activity includes activity to bind to at least one transcription factor.

As used herein, “nucleotide” refers to any naturally occurring nucleotide and non-naturally occurring nucleotide. “Derived nucleotide” refers to a nucleotide which is different from naturally occurring nucleotides but has a function similar to that of its original naturally occurring nucleotide. Such derived nucleotides are well known in the art.

As used herein, “variant” refers to a substance, such as polynucleotide, or the like, which differs partially from the original substance. Examples of such a variant include a substitution variant, an addition variant, a deletion variant, a truncated variant, an allelic variant, and the like. Allele refers to a genetic variant located at a locus identical to a corresponding gene, where the two genes are distinguished from each other. Therefore, “allelic variant” refers to a variant which has an allele relationship with a certain gene. “Homolog” of a nucleic acid molecule refers to a nucleic acid molecule having a nucleotide sequence having homology with the nucleotide sequence of a reference nucleic acid molecule. Representatively, “homolog” refers to a polynucleotide which hybridizes to a reference nucleic acid molecule under stringent conditions. In the case of the nucleic acid molecule of the present invention, a “homolog” is a nucleic acid molecule having a nucleic acid sequence having homology with the nucleic acid sequence of the decoy of the present invention, whose biological function is the same as or similar to the promoter of the present invention. Therefore, the concepts of “homolog” and variant” overlap partially. Therefore, a homolog has amino acid or nucleotide homology with a certain gene in a certain species (preferably at least 60% homology, more preferably at least 80%, at least 85%, at least 90%, and at least 95% homology). A method for obtaining such a homolog is clearly understood from the description of the present specification. For example, a homolog of the decoy of the present invention may be a homologous gene in the same species or a corresponding gene in other species. Therefore, the decoy of the present invention may include all homologs of the decoy.

### DETAILED DESCRIPTION OF THE INVENTION

According to a first aspect of the present invention, a pharmaceutical composition for treating and preventing diseases and disorders associated with an ischemic condition of the brain, and disorders caused by the diseases and the disorders, and a method of using the composition for treating and preventing diseases and disorders associated with an ischemic condition of the brain, and disorders caused by the diseases and the disorders, are provided. The composition comprises at least one NF-κB decoy, and a pharmaceutically acceptable carrier.

In one embodiment, the disease targeted by the present invention may be at least one disease selected from the group consisting of subarachnoid hemorrhage, hypertensive intracerebral hemorrhage, cerebral infarct, brain ischemia, brain tumor, head injury, chronic subdural hemorrhage, and acute subdural hemorrhage. In another embodiment, the disease and the disorder caused by the diseases and the disorders associated with the above-described ischemic condition of the brain is selected from the group consisting of neuropathy, motor disorders, intelligence disorder, dementia, partial paralysis, headache, and incontinence of urine.

In another embodiment, the pharmaceutically acceptable carrier may be any pharmaceutical acceptable carrier, preferably liposome. More preferably, the pharmaceutical composition of the present invention may be provided in the form of HVJ-liposome.

The NF-κB decoy in the present invention comprises a sequence GGATTTCCC. More preferably, the NF-κB decoy may comprise CCTTGAAGGGATTTCCCTCC (SEQ ID NO: 1). In another embodiment, the NF-κB decoy may be further modified.

In a preferred embodiment, the composition and method of the present invention may be administered through an administration route including a carotid artery.

In another aspect, the present invention provides a method for gene transfection through a route other than administration direct to the brain, and a composition for that purpose. This composition comprises at least one decoy and a pharmaceutically acceptable carrier.

In a preferred embodiment, the route other than administration direct to the brain is infusion to a carotid artery. Any gene may be appropriate for transfection through the route other than administration direct to the brain. Preferably, such a gene may exhibit an effect due to its expression in the brain. Examples of the gene include decoys for NF-κB, STAT-1, GATA-3, STAT-6, AP-1, E2F, Ets, and CRE. Examples of preferable decoys include 5′-CCTTGAAGGGATTTCCCTCC-3′ (SEQ ID NO: 1)(NF-κB decoy), 5′-GATCTAGGGATTTCCGGGAAATGAAGCT-3′ (SEQ ID NO: 2) (STAT-1 decoy), 5′-AGCTTGAGATAGAGCT-3′ (SEQ ID NO: 3) (GATA-3 decoy), 5′-GATCAAGACCTTTTCCCAAGAAATCTAT-3′ (SEQ ID NO: 4) (STAT-6 decoy), 5′-AGCTTGTGAGTCAGAAGCT-3′ (SEQ ID NO: 5) (AP-1 decoy), and 5′-AATTCACCGGAAGTATTCGA-3′ (SEQ ID NO: 6) (Ets decoy), 5′-TGACGTCA-3′ (CRE decoy), or oligonucleotides containing complements thereof, mutants thereof, or compounds containing them therein. In a preferred embodiment, the pharmaceutically acceptable carrier may be a liposome.

The present invention provides evidence that in vivo transfection of cis-element decoy, to which the transcription factor NF-κB is: linked, attenuated neuronal damage after global brain ischemia.

In one embodiment, NF-κB decoy ODNs are successfully introduced into the nuclei of neurons by injecting them through a carotid artery and across a blood-brain barrier. The transfected NF-κB decoy ODNs were assessed on immunoreactivity using TUNEL labeling (DNA fragmentation) and MAP2 (neuronal marker). As a result, the inventors demonstrated that the transfected NF-κB decoy ODNs suppressed gene expression related to NF-κB signals in the hippocampus and attenuated neuronal damage caused by global brain ischemia. Therefore, in the present invention, the transfection of neurons with the NF-κB decoy ODNs through a carotid artery provides a novel strategy to protect the brain against ischemic injury during global brain ischemia.

Conventionally, brain ischemia has been treated by deep hypothermia. The deep hypothermia is a basic strategy for brain protection during circulatory arrest which reduces the cerebral energy requirements. However, deep hypothermic circulatory arrest carries an adverse risk of neuronal damage, and is associated with complications (seizures, cerebral palsy, motor dysfunction, memory deficits or the like) (see Rappaport L A, Wypij D, Bellinger D C, Helmers S L, Holmes G L, Barnes P D, et al., Circulation. 1998; 97:773-9; Bellinger D C, Jonas R A, Rappaport L A, Wypij D, Wernovsky G, Kuban K C, et al., N Engl J Med. 1995; 332:549-55; and Reich D L, Uysal S, Sliwinski M, Ergin M A, Kahn R A, Konstadt S N, et al., J Thorac Cardiovasc Surg. 1999; 117:156-63). Neuronal damage (including necrosis and delayed neuronal death) is one cause of these neurological injuries. In treatment according to the present invention, no neurological event was revealed in rats, and histological study showed no infarction area in the brain section. The present inventors concluded that the rats 7 days after ischemia in a control group may have had possibly impaired learning ability compared with those in a NF-κB decoy group, although all rats survived. In addition to deep hypothermia, a number of methods have been reported concerning attenuation of neuronal damage (Aoki M, Jonas R A, Nomura F, Stromski M E, Tsuji M K, Hickey P R, et al. J Thorac Cardiovasc Surg. 1994; 108:291-301). However, these reports have mainly focused on regulation of energy requirements and metabolism, and they have generally failed to prove clinical success. Therefore, there has been a demand for alternative method on the basis of other mechanisms (regulation of gene expression related to ischemia-reperfusion injury, and the like). The method of the present invention solved such a problem.

Recent reports have demonstrated that apoptosis may play an important role in delayed neuronal damage after circulatory arrest (see Cheng Y, Deshumukh M, D'Costa M, Demaro J A, Gidday J M, Shah A, et al., J Clin Invest 1998; 101:1992-9; and Kurth C D, Priestley M, Golden J, McCann J, Raghupathi R., J Thorac Cardiovasc Surg. 1999; 118:1068-77). A number of molecular signals (including inflammation-related cytokines and adhesion molecules) are involved in apoptosis. These inflammation-related factors are upregulated mainly by transcriptional activation of NF-κB. This NF-κB is an oxidation stress reactive molecule. Whether or not regulation of cytokine mRNA (TNF-α, IL-1β, and the like) level directly blocks neuronal damage is not clear. However, at a minimum, these inflammatory cytokines are responsible for ischemia-related neuronal damage. The present invention demonstrated that NF-κB may play an important role in attenuation of ischemia-reperfusion injury and neuronal damage after global brain ischemia. TUNEL staining is a non-specific technique which may show DNA injury and DNA repair, and may even be positive in necrotic cells. Therefore, other tests were further conducted. In the present invention, histological experiments demonstrated that TUNEL-positive neurons were about 15% in total neurons (less than TUNEL-positive neurons 7 days after global brain ischemia) 2 days after global brain ischemia. Neuronal damage (including both necrosis and delayed neuronal death) occurred at least between 2 days and 7 days after global brain ischemia.

In addition, NF-κB has been speculated to function through a number of pathways, and these pathways may also be associated with neuronal damage after global brain ischemia. These mechanisms are as follows: (1) activation of NF-κB partially mediates free radical damage in a number of tissues (including the brain) (see Schreck R, Rieber P, Baeuerle P A., EMBO J. 1991; 10:2247-58); (2) activation NF-κB seems to cause glutamate cytotoxicity (Grilli M et al., 1996, supra.); (3) NF-κB functions in upregulation of inducible nitric oxide synthase and cyclooxygenase 2 (see Schulze-Osthoff K, Ferrari D, Riehemann K, Wesselborg S., Immunobiology. 1997;198:35-49); and (4) NF-κB may mediate activation of the CD95 ligand which causes delayed neuronal damage (see Vogt M, Bauer M K, Ferarri D, Schulze-Osthoff K., FEBS Lett. 1998;429:67-72). These mechanisms are all potential targets of the NF-κB decoy ODN method. Therefore, the present invention provides a composition and method for treating and preventing neuronal damage by acting on at least one of the above-described mechanisms.

Further, target genes for NF-κB include apoptosis-related genes (including TP53 (see Wu H, Lozano G., J Biol Chem. 1994, 269:20067-74) and c-myc (LaRosa F A, Pierce J W, Sonenshein G E., Mol Cell Biol. 1994; 14:1039-44)). Therefore, a gene therapy according to the present invention using decoy ODNs against the NF-κB binding site suppresses gene expression relating to inflammatory response and the subsequent neuronal damage (including apoptosis), thereby providing a novel strategy for neuronal protection during ischemia.

A number of researchers have reported strategies using gene therapy for brain protection (see Hagihara Y, Saitoh Y, Kaneda Y, Kohmura E, Yoshimine T., Gene Ther. 2000; 7:759-63; and Ono S, Date I, Onoda K, Shiota T, Ohmoto T, Ninomiya Y, et al., Hum Gene Ther. 1999; 10:335). In these reports, genes were infused into the subarachnoid space or brain ventricle. However, no study has shown effective gene transfection by means of infusion through a carotid artery. This is because the blood-brain barrier prevents the entry of a number of foreign substances and microorganisms. During and after global brain ischemia, however, permeability across the blood-brain barrier increases; in fact, relief of the blood-brain barrier has been reported to extend for up to 6 hours after ischemia (see Preston E, Foster D O., Brain Res. 1997:761:4-10). The present inventors believe that the present inventors' success in transfecting neurons with the decoy ODNs was due to accumulation of HVJ-liposomes complex in brain tissues. To the best of the present inventors' knowledge, this is the first report of successful gene transfection through a carotid artery and across the blood-brain barrier. Such an effect provides a safer and more comfortable therapy to patients which have not been achieved by conventional techniques.

Decoy therapy has a number of benefits (including immediate effect, low cost, and substantially no complications). Gene therapy using naked E2F decoy has already been attempted in clinical settings to prevent vein graft disease (see Mann M J. Whittemore A D, Donaldson M C, Belkin M, Conte M S, Polak J F et al., Lancet. 1999:354:1493-8). However, in the present invention, since transfection of naked ODN has limitations in its efficiency in the brain through a carotid artery, more safe vectors may be utilized. Therefore, clinical application of NF-κB decoy therapy through a carotid artery using a HVJ-liposome or other vectors may be possibly attempted for brain protection against ischemic injury during circulatory arrest. NF-κB decoy ODN therapy through vessels has a potential of wide application in clinical use for brain protection (retrograde reperfusion for cerebroplegia).

In summary, the results of the present invention indicate that administration of NF-κB decoy ODNs during global brain ischemia attenuates neuronal damage in the hippocampus CA1 region in a rat model. Thus, NF-κB decoy ODN administration through a carotid artery can protect neurons during global brain circulatory arrest, raising the possibility that NF-κB decoy ODNs may become a promising therapeutic and pharmaceutical agent for protecting the brain against global ischemia. The present invention demonstrated that this method of gene transfection to the brain is applicable to not only cardiac surgery but also in other fields, such as neurological surgery and brain surgery. Thus, the demonstration of the decoy applications in the cranial nerve field is a significant effect, and the usefulness thereof is almost beyond description.

Hereinafter, the present invention will be described by way of examples, and the following examples are provided only for illustrative purposes. Therefore, the scope of the present invention is limited only by the claims, but not the examples.

### EXAMPLES

### Example 1

### Preparation of HVJ Virus-Liposome Complex

A HVJ-liposome complex was prepared as described in references (Morishita R, Sugimoto T, Aoki M, Kida I, Tomita N, Moriguchi A, et al., Nat Med. 1997; 3:894-9). Briefly, phosphatidylserine (PS), phosphatidylcholine (PC) and cholesterol (Chol) were mixed in a weight ratio of 1:4.8:2. The lipid mixture (10 mg) was deposited on the sides of a flask by removal of a solvent (tetrahydrofuran) in a rotary evaporator. Dried lipid was hydrated in 200 μl of physiological saline containing 200 μg of ODN. Liposomes were prepared by shaking and sonication. Liposome suspension (0.5 mL, containing 10 mg of lipids) was mixed with HVJ (10,000 hemagglutinating units) inactivated in physiological saline having a total volume of 4 mL. The mixture was incubated at 4° C. for 5 minutes and then for 30 minutes with gentle shaking at 30° C. Free HVJ was removed from the HVJ-liposomes by density gradient centrifugation. The top layer of the sucrose gradient was collected for use. The sequences of phosphorothioate ODN are the following: NF-κB decoy ODN, 5′-CCTTGAAGGGATTTCCCTCC-3′ (SEQ ID NO: 1), and 3′-GGAACTTCCCTAAAGGGAGG-5′ (SEQ ID NO: 7); and scrambled decoy ODN, 5′-TTGCCGTACCTGACTTAGCC-3′ (SEQ ID NO: 8) and 3′-AACGGCATCCACTGAATGGG-5′ (SEQ ID NO: 9).

### Example 2

### Preparation of Global Brain Ischemia Model and Evaluation of the Model

The present inventors established a rat global brain ischemia-reperfusion model using a modified occlusion technique for a subclavian-carotid artery (Torre J C, Fortin T., Brain Res Bull. 1991; 26:365-72). 300 g to 500 g-weight male Sprague-Dawley rats were used. All of the animals were cared for in accordance with “Guide for the Care and Use of Laboratory Animals” prepared by the Institute of Laboratory Animal Resources in the Osaka University Medical School. Each rat was anesthetized by intraperitoneal administration of 50 mg/kg pentobarbital, and intubated into the mouth. A rodent ventilator was set at 10 mL/kg volume and 50 to 60 strokes/min to maintain a P_{CO2} of 35 mmHg. During the experiment, the rats were warmed at 36° C. using a heating blanket, except for the brain. After thoracotomy, the left lobe of the thymus was removed. The aortic arch was identified, and the innominate artery, left common carotid artery, and left subclavian artery were snared by 50 nylon sutures. The right common carotid artery was exposed in the neck, and cannulated with a polyethylene tube (PE10, Becton Dickinson Company, Franklin Lakes, N.J.). Global brain ischemia was induced by clamping all 3 sutured arteries for 20 minutes.

### Example 3

### NF-κB Decoy Oligodeoxynucleotide Transfection in Brain Ischemia

Immediately after clamping the arteries, the HVJ-liposome complex containing either of NF-κB decoy ODNs (NF decoy group) or scrambled decoy ODNs (S decoy group) was infused into the right carotid artery to perfuse the brain tissue. These drugs were stored at 4° C., and 2 mL per animal was infused. In this procedure, the pharyngeal temperature fell from 35.2° C.±0.2° C. to 33.1° C.±0.5° C. No neurological events were observed in any animal after the surgical procedures.

Global ischemia-reperfusion brains were evaluated by three methods. First, three rats were killed one hour after reperfusion, and brain sections were observed with fluorescence microscopy to investigate transfection of fluorescein isothiocyanate (FITC)-labeled ODN delivery. Second, five rats from each group were killed one hour after reperfusion, and the hippocampus, including the CA1 region, were collected to test the effect of the transfected NF-κB decoy ODNs on expression of messenger RNAs which are known to be activated by NF-κB. The samples weighed of 20 mg to 25 mg after blood vessels were stripped away. Third, 10 rats from each group were killed seven days after global brain ischemia for histological study by means of terminal deoxynucleotidyl transferase-mediated deoxyuridine nick-end labeling (TUNEL) staining or histochemical analysis by immunohistochemistry with microtubule-associated protein 2 (MAP2), in order to investigate neuronal damage.

(In Vivo Transfection of NF-κB Decoy ODN Through Carotid Artery During Global Brain Ischemia)

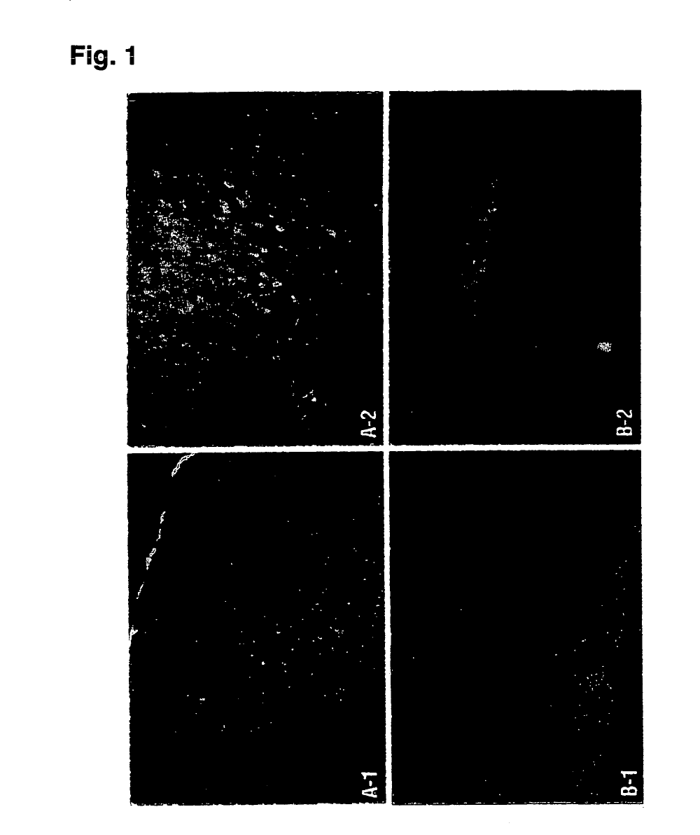

In the present inventors' preliminary study, the present inventors infused naked FITD-labeled NF-κB ODNs into a carotid artery without any vectors during global brain ischemia. However, fluorescence was not detected in the brain tissue by this method (data not shown). Thereafter, the present inventors tried using the HVJ-liposome method to transfect NF-κB decoy ODNs into brain tissue. One hour after reperfusion, in all of the rats examined, the present inventors observed transfection of cells with FITC-labeled ODNs not only in the intima of arteries but also in neurons (particularly, neurons in the cortex and hippocampus) (FIG. 1). Fluorescence was localized mainly in cell nuclei. Therefore, in the present inventors' model, NF-κB decoy ODNs could be transfected into the brain tissue through the blood-brain barrier during global brain ischemia.

(Results)

The present inventors clearly succeeded in introducing NF-κB decoy oligodeoxynucleotide through a carotid artery into rat cranial nerve in global brain ischemia. Polymerase chain reaction showed that transfected NF-κB decoy oligodeoxynucleotide effectively inhibited expression of mRNAs for tumor necrosis factor α, interleukin 1β and intracellular adhesion molecules 1 one hour after global brain ischemia. Terminal deoxynucleotidyl transferase-mediated deoxyuridine nick-end labeling staining and immunohistochemistry using microtubule-associated protein 2 showed transfected NF-κB decoy oligodeoxynucleotide significantly attenuated neuronal damage seven days after global brain ischemia.

(Quantification of TNF-α, IL-1β and ICAM-1 mRNAs in Hippocampus)

Next, in this example, TNF-α, IL-1β and ICAM-1 mRNAs were quantitated in the hippocampus.

The present inventors employed a real-time polymerase chain reaction (PCR) system to investigate the effect of in vivo transfection of NF-κB decoy ODNs versus S decoy control ODNs on the expression of genes which are known to respond to the signal of NF-κB one hour after reperfusion. With this technique, complementary DNA amplification was quatitated. The technique includes fluorescence-based real-time PCR followed by measurement of amplification using the ABI PRISM 7700 Sequence Detection System (Biosystems, Foster City, Calif., USA).

In brief, total RNA was purified from each 20- to 25-mg hippocampus sample using the RNA easy Mini Kit (Qiagen, Hilden, Germany) in accordance with the manufacturer's instructions. The RNA samples were frozen in liquid nitrogen and stored at −80° C. until use. To test for gene transcription, 2 μg of RNA was reverse-transcribed using RNase-H-negative Moloney's murine leukemia virus reverse transcriptase (SUPERSCRIPT 2, Gibco BRL, Life Technologies, Inc, Rockville, Md.) in a total volume of 40 μL, as recommended by the manufacturer. One eightieth of the cDNA was used for each PCR, and measurement of each transcription was performed in triplicate. The technique of real-time PCR is based on hydrolysis of a specific fluorescence probe at each amplification cycle by the 5′-endonuclease activity of Taq polymerase. This technique was performed as described in Depre et al. (Depre C, Shipley G L, Chen W, Han Q, Doenst T, Moore M, et al., Nat Med. 1998; 4:1269-75), with some modification. The nucleotide sequences of the forward primers, reverse primers, and probes were as follows:

TNFα,

(SEQ ID NO: 10)

| | |
|---|---|
| forward primer | CCACCACGCTCTTCTGTCTACT, |

(SEQ ID NO: 11)

| | |
|---|---|
| reverse primer | TTGGTGGTTTGGGACGACGT, |
| and | |

(SEQ ID NO: 12)

| | |
|---|---|
| probe | CCCAGACCCTCACACTCAGATCATCTTC; |
| IL-1β, | |

(SEQ ID NO: 13)

| | |
|---|---|
| forward primer | CCACCTCAATGGACAGAACATAAG, |

(SEQ ID NO: 14)

| | |
|---|---|
| reverse primer | GACAAACCGCTTTTCCATCTTC, |
| and | |

(SEQ ID NO: 15)

| | |
|---|---|
| probe | CAAGGAGAGACAAGCAACGACAAAATCCC; |
| and | |
| ICAM-1, | |

(SEQ ID NO: 16)

| | |
|---|---|
| forward primer | TTCAAGCTGAGCGACATTGG, |

(SEQ ID NO: 17)

| | |
|---|---|
| reverse primer | TCAGTGTCTCATTCCCAAGCA, |
| and | |

(SEQ ID NO: 18)

| | |
|---|---|
| probe | TCTGCCACCATCACTGTGTATTCGTTCC. |

For each molecule assayed here, the primer pair, or at least one primer or probe, was designed to span over at least one intron so that only mRNA would be measured. In fact, when genomic DNA was used as a target, no signal was detected for any of the molecules. Primers and probes were used at 200 mmol/L in each PCR with 50 cycles of a 15-second denaturing step at 95° C. and a 1-minute annealing step at 60° C. The correlation coefficient of standard curves generated in each measurement was always 0.97 or better, and the coefficient of variation in the triplicate samples was usually no more than 10%. Because of the relative lack of precision in the measurement of RNA concentration using spectrophotometry, the level of transcripts for the cellular enzyme glyceraldehydes 3-phosphate dehydrogenase (GAPDH) was quantitatively measured in each sample as the internal control. The GAPDH primer and probe sequences were as follows:

(SEQ ID NO: 19)

| | |
|---|---|
| forward primer, | CCATCACTGCCACTCAGAAGAC; |

(SEQ ID NO: 20)

| | |
|---|---|
| reverse primer, | TCATACTTGGCAGGTTTCTCCA; |
| and | |

(SEQ ID NO: 21)

| | |
|---|---|
| probe, | CGTGTTCCTACCCCCAATGTATCCGT. |

The mRNA/GAPDH value was calculated for each sample, and then the induction value compared with the normal rat mRNA/GAPDH level was calculated.

(Results)

(TNF-α, IL-1β and ICAM-1 mRNA Expression in Hippocampus)

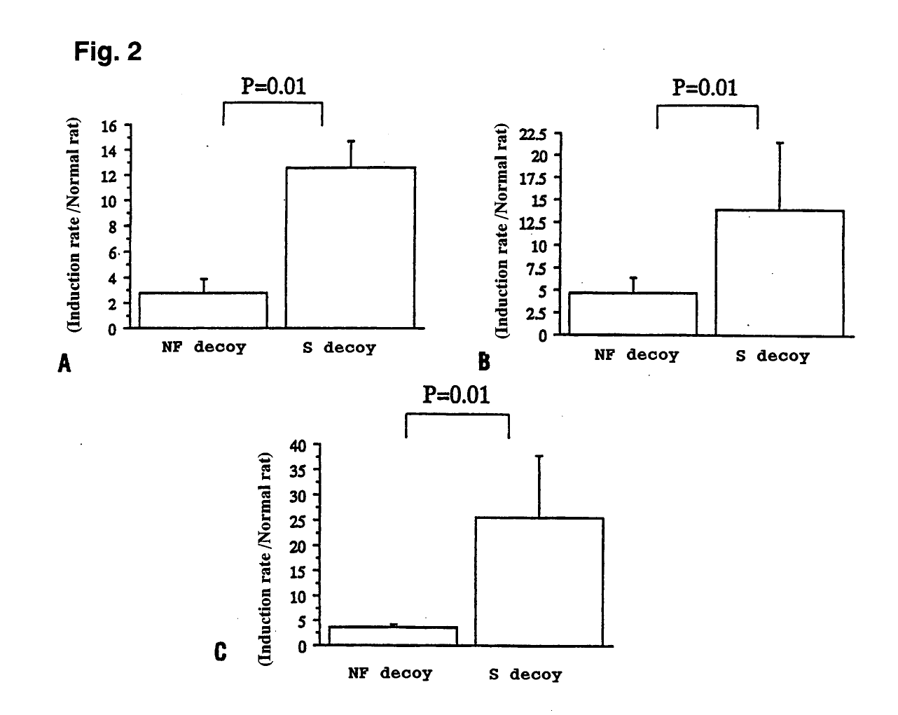

In the NF decoy group, the fold-induction rate of expression of the gene encoding TNF-α one hour after reperfusion compared with that seen in normal cells was 2.8±11.1, whereas the fold-induction rate of the S decoy group was 12.5±2.2. The fold-induction rates of IL-1β and ICAM-1 mRNA expression one hour after reperfusion were 4.7±1.7 and 3.5±0.5 in the NF decoy group and 14.0±7.5 and 25.7±12.0 in the S decoy group, respectively (FIG. 2). The expression of these three genes was activated by NF-κB, and was effectively suppressed by the transfection of NF-κB decoy ODNs through a carotid artery (P=0.01, P=0.01 and P=0.1, respectively). These data demonstrated that the transfected NF-κB decoy ODNs effectively blocked gene expression related to NF-κB in ischemia-reperfusion injury in the hippocampus.

### Example 4

### Blockade of Neuronal Damage by NF-κB Decoy ODNs in the Hippocampus CA1 Region

Next, in this example, blockade of neuronal damage by NF-κB decoy ODNs in the hippocampus CA1 region was histochemically evaluated.

(TUNEL Staining and Immunohistochemstry)

It is known that in normal mice one week after global brain ischemia, ischemic damage can be detected, particularly in the CA1 region of the hippocampus, and the number of TUNEL-positive neurons increases (Jonas R A. Hypothermia, circulatory arrest, and the pediatric brain. J Cardiothorac Vasc Anesth. 1996; 10:66-74). The presence of TUNEL-positive neurons does not directly reveal the occurrence of apoptosis, but does indicate DNA damage. The expression levels of MAP2 (cytoskeletal protein is a marker for ischemic injury, and its expression is reduced) have been observed after global brain ischemia (Vanickey I, Baichen T, Diemer N H., Neuroreport. 1995; 7:161-4). To assess the effect of transfection of NF-κB decoy ODNs on neuronal ischemic injury, brains were quickly frozen in liquid nitrogen and sectioned coronally through the rostrocaudal extent of the hippocampus. For TUNEL staining, 5-μm sections were fixed in 1% paraformaldehyde. TUNEL staining was performed using the ApopTag In Situ Apoptosis Detection Kit (Intergen Co, Purchase, N.Y.) as recommended by the manufacturer. The reaction product was visualized by development with 3,3′-diaminobenzidine and H₂O₂. The brain sections stained by TUNEL were also stained using hematoxylin and eosin. Thereafter, the percentage of the total number of neurons that were TUNEL-positive was calculated in the CA1 region (500 μm in length) in three sections in each rat.

Immunohistochemistry was performed on the brain sections using the avidin-biotin peroxidase system (ABC kit; Vector Laboratories, Inc, Burlingame, Calif.). Five-micrometer sections were fixed in 2% paraformaldehyde and incubated with a monoclonal MAP2 antibody (Upstate Biotechnology, Lake Placid, N.Y.) overnight at 4° C. The sections were stained using the ABC immunological peroxidase system according to the manufacturer's recommendations. The reaction product was visualized by development with 3,3′-diaminobenzil and H₂O₂, and these sections were also stained using hematoxylin and eosin. The number of MAP2-positive neurons was counted in the CA1 region (500 μm in length) in 3 sections in each rat.

(Statistical Analysis)

Data are presented as means±standard deviation. Statistically significant differences between the two groups was calculated using a Mann-Whitney U test.

(Results)

(Blockade of Neuronal Damage NF-κB Decoy ODNs in the Hippocampus CA1 Region)

Next, brain tissue was evaluated histologically seven days after global brain ischemia to determine the protective effect of NF-κB decoy ODNs against neuronal damage. TUNEL-positive neurons were detected in both hemispheres, and neuronal damage was estimated in the right hemisphere.

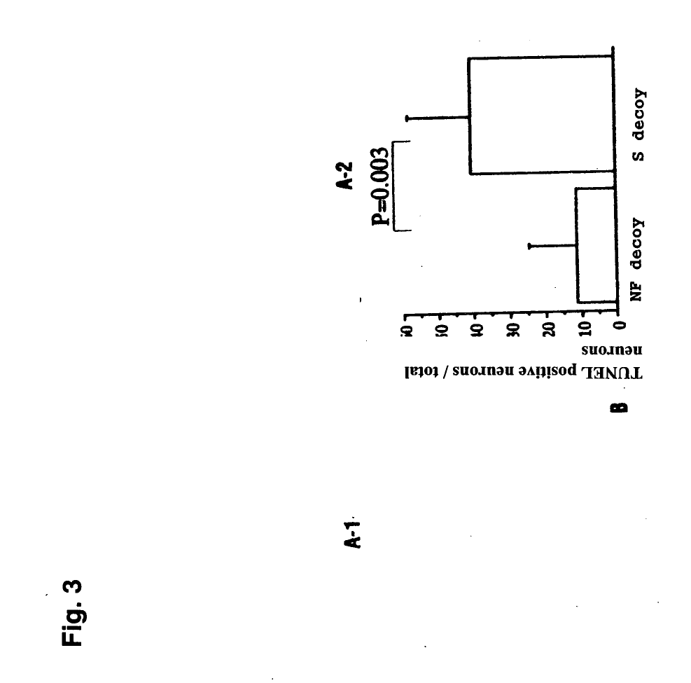

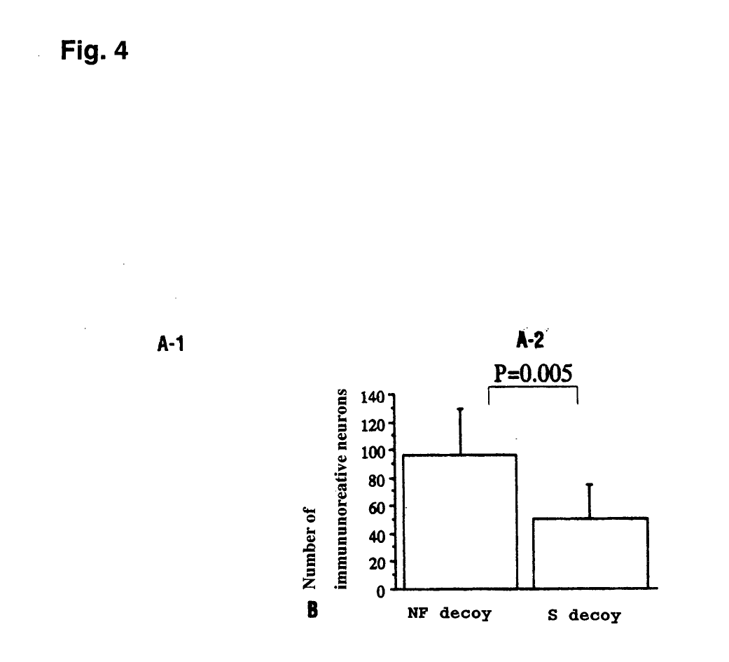

In the NF decoy group, 7 days after global brain ischemia, there were fewer TUNEL-positive neurons, compared with the S decoy group (11.3%±13.1% in the NF decoy group and 40.3%±18.0% in the S decoy group, P=0.003; FIG. 3). The number of MAP2-positive neurons were higher in the NF decoy group than in the S decoy group (96.4±33.0 cells/500 μm long in the NF decoy group and 50.6±23.8 cells/500 μm long in the S decoy group, P=0.005; FIG. 4). These data show that the transfection of NF-κB into neurons through a carotid artery attenuated neuronal damage after global brain ischemia.

(Conclusion)

The therapeutic transfection of NF-κB decoy oligodeoxynucleotide in brain ischemia is effective for attenuation of neuronal damage as well as protection of cerebrum and nerve in brain ischemia.

### Example 5

### Intracerebral Gene Transfection of Other Decoys into Carotid Artery

Next, in order to demonstrate that other genes pass across the blood-brain barrier and thereafter transfect the brain. As examples, STAT-1 decoy (5′-GATCTAGGGATTTCCGGGAAATGAAGCT-3′ (SEQ ID NO: 2)), GATA-3 decoy (5-AGCTTGAGATAGAGCT-3′ (SEQ ID NO: 3)), STAT-6 decoy (5′-GATCAAGACCTTTTCCCAAGAAATCTAT-3′ (SEQ ID NO: 4)), AP-1 decoy (5′-AGCTTGTGAGTCAGAAGCT-3′ (SEQ ID NO: 5)) and Ets decoy (5′-AATTCACCGGAAGTATTCGA-3′ (SEQ ID NO: 6)) were used.

For the above-described decoys, HVJ virus-liposome complexes were prepared in accordance with the description in Example 1.

The present inventors infused each of the above-described naked FITC-labeled decoy ODN into a carotid artery during global brain ischemia, without any vector. However, when this method was used, no fluorescence was detected in brain tissue (data not shown). Next, the present inventors tried to use a HVJ-liposome method for transfecting each of the above-described decoy ODN into brain tissue. One hour after reperfusion, the present inventors observed the transfection of FITC-labeled ODN into cells in all of the tested rats at not only the intima of arteries but also neurons (particularly, cortex and hippocampus neurons) (data not shown). Fluorescence was localized mainly in cell nucleus. Therefore, in the present inventors' model, brain tissue in global brain ischemia could be transfected with the above-described decoy ODN other than NF-κB decoy ODN across the blood-brain barrier.

In this example, the present invention demonstrated the possibility that any decoy can pass across the blood-brain barrier and transfect the brain.

### INDUSTRIAL APPLICABILITY

A pharmaceutical composition for treating or preventing a disease or a disorder caused by ischemia in the brain using a decoy is provided. The pharmaceutical composition of the present invention achieved gene transfection in the brain by administration at sites other than the brain. Thus, the present invention may provide a non-invasive and repeatable method for treating and preventing a brain disease or disorder.

1. A pharmaceutical composition for treating and preventing a disease and a disorder associated with an ischemic condition of a brain, and a disease and a disorder caused by the disease and the disorder, the composition comprising:
at least one NF-κB decoy; and
a pharmaceutically acceptable carrier.

2. A composition according to claim 1, wherein the disease is at least one disease selected from the group consisting of subarachnoid hemorrhage, hypertensive intracerebral hemorrhage, cerebral infarct, brain ischemia, brain tumor, head injury, chronic subdural hemorrhage, and acute subdural hemorrhage.

3. A composition according to claim 1, wherein the disease and the disorder caused by the disease and the disorder associated with the ischemic condition of the brain is selected from the group consisting of neuropathy, motor disorders, intelligence disorder, dementia, partial paralysis, headache, and incontinence of urine.

4. A composition according to claim 1, wherein the pharmaceutically acceptable carrier is a liposome.

5. A composition according to claim 1, wherein the NF-κB decoy comprises a sequence GGATTTCCC.

6. A composition according to claim 1, wherein the composition is appropriate to an administration route including a carotid artery.

7. A composition for carrying out gene transfection in a brain by a route other than direct administration to the brain, the composition comprising:
at least one decoy; and
a pharmaceutically acceptable carrier.

8. A composition according to claim 7, wherein the route other than direct administration to the brain is an infusion to a carotid artery.

9. A composition according to claim 7, wherein the decoy is NF-κB.

10. A composition according to claim 7, wherein the pharmaceutically acceptable carrier is a liposome.

11. A method for treating and preventing a disease and a disorder associated with an ischemic condition of a brain, and a disease and a disorder caused by the disease and the disorder, the method comprising the step of:
administering a composition to a subject,
wherein the composition comprises:
at least one NF-κB decoy; and
a pharmaceutically acceptable carrier.

12. A method according to claim 11, wherein the disease is at least one disease selected from the group consisting of subarachnoid hemorrhage, hypertensive intracerebral hemorrhage, cerebral infarct, brain ischemia, brain tumor, head injury, chronic subdural hemorrhage, and acute subdural hemorrhage.

13. A method according to claim 11, wherein the disease and the disorder caused by the disease and the disorder associated with the ischemic condition of the brain is selected from the group consisting of neuropathy, motor disorders, intelligence disorder, dementia, partial paralysis, headache, and incontinence of urine.

14. A method according to claim 11, wherein the pharmaceutically acceptable carrier is a liposome.

15. A method according to claim 11, wherein the NF-κB decoy comprises a sequence GGATTTCCC.

16. A method according to claim 11, wherein the composition is appropriate to an administration route including a carotid artery.

17. A method for carrying out gene transfection in a brain by a route other than direct administration to the brain, the method comprising the step of:
administering a composition into the route other than the direct administration to the brain,
wherein the composition comprises, in an appropriate form:
at least one decoy; and
a pharmaceutically acceptable carrier.

18. A method according to claim 17, wherein the route other than direct administration to the brain is an infusion to a carotid artery.

19. A method according to claim 17, wherein the decoy is NF-κB.

20. A method according to claim 17, wherein the pharmaceutically acceptable carrier is a liposome.
